(19) United States
(12) Patent Application Publication
(10) Pub. No.: US 2004/0054364 A1
(43) Pub. Date: Mar. 18, 2004

(54) Ultrasonic surgical instrument

(76) Inventors: Ernest Aranyi, Easton, CT (US); Douglas J. Cuny, Bethel, CT (US); Russell Heinrich, Madison, CT (US); Amit Lal, Ithica, NY (US); Bill Lewis, Monroe, CT (US); Philip C. Roy, Hamden, CT (US)

Correspondence Address: Paul R Audet, Tyco Healthcare Group, Senior Patent & Trademark Counsel, 150 Glover Avenue, Norwalk, CT, 06856, US

(21) Appl. No.: 10/467,512

(22) PCT Filed: Feb. 8, 2002
(86) PCT No.: PCT/US02/04988

(51) Int. Cl. A61B 18/04

(52) U.S. Cl. 606/27; 606/28

(57) ABSTRACT

An ultrasonic surgical instrument is provided which includes a handle assembly, a body extending distally from the handle assembly and an end effector configured to effect cutting, dissection, coagulation and/or ligation of tissue. The end effector includes an ultrasonic member. A transducer is supported adjacent, on or within the ultrasonic member and is connected to a power source. Upon actuation of the power source, the transducer effects vibration of the ultrasonic member. In one preferred embodiment, the end effector is mounted for articulation about the distal end of the instrument.

This application claims priority to U.S. Provisional Application Serial No. 60/267,251, filed Feb. 8, 2001, which is incorporated herein by reference in its entirety.

### BACKGROUND

1. Technical Field

The present disclosure relates generally to ultrasonic surgical instruments. More specifically, the present disclosure relates to ultrasonic surgical instruments having an end effector configured to effect tissue dissection, cutting, coagulation, ligation and/or hemostatis and having a microelectromechanical system incorporated therein (“MEMS”), which instrument can be used in open as well as laparoscopic or endoscopic surgical procedures.

2. Background of Related Art

Ultrasonic instruments for surgical use are well known and are used in a variety of surgical procedures for dissecting, cutting, ligating, effecting coagulation in, and/or effecting hemostasis in tissue. Typically, ultrasonic surgical instruments include a handpiece for grasping the instrument, a transducer attached to the proximal end of the handpiece, and a vibration coupler extending from the transducer through a body of the instrument to an end effector of the instrument. The transducer generates vibrations in the ultrasonic frequency range which are transmitted from the handpiece of the instrument to the end effector via the vibration coupler. This configuration, although effective in some applications, has several drawbacks. For example, the power of the instrument is attenuated when ultrasonic energy is transmitted from a proximal end of a device to a distal end of the device. Further, power losses are enhanced at couplings and seals of the instrument. As such, a large, heavy transducer is required to operate known surgical instruments. Moreover, contact between the vibration coupler and stationary components of the instrument result in mechanical faults in the instrument. Finally, the vibration coupler acts as a pump which draws bodily fluids from the distal end of the instrument to the proximal end of the instrument thereby making sterilization of the instrument after use difficult.

The use of an elongated vibration coupler also limits the operational features of the instrument available to a surgeon. More specifically, because the vibration coupler transmits vibrations from the transducer to the end effector, the inclusion of an articulation joint into the vibration coupler is difficult and inefficient. Accordingly, known ultrasonic instruments typically do not include articulating end effectors. Moreover, because the vibrations are transmitted from the transducer at the proximal end of the instrument to the distal end of the instrument, along a stiff vibration coupler, e.g. an elongated titanium rod, vibration energy is transmitted primarily along the rod in longitudinal waves. Any transverse vibrations that do occur as the energy is transmitted along the length of the vibration coupler reduces the overall effeciency of the system.

### SUMMARY

An ultrasonic surgical system is provided which includes a sugical instrument having an end effector with a transducer, a control module and a conductive cable interconnecting the surgical instrument to the control module. The control module is adapted to be connected to a power source, which may include an electrical outlet, an a/c generator, or a battery pack, etc., and includes control circuitry to drive the transducer positioned on the end effector of the instrument at an ultrasonic frequency or multiple ultrasonic frequencies independently or sinultaneously. Alternately, the control circuitry may be incorporated into the power source. The ultrasonic instrument includes a handle assembly, a body portion and an integral or removable end effector configured to effect cutting, dissection, ligation, hemostasis and/or coagulation of tissue. The end effector includes an ultrasonic member which is preferably formed from a silicon composite, e.g., silicon-titanium composite material. The transducer is supported on, within or adjacent the ultrasonic member of the end effector. The ultrasonic member may have a variety of different configurations including different hook configurations, rectangular, circular, square, etc. The end effector may also include a clamp member or shear probe. In one preferred embodiment, the endoscopic body portion of the instrument is rotatable about its longitudinal axis to effect rotation of the end effector about the longitudinal axis of the endoscopic body portion. Alternately, the end effector or ultrasonic member may be rotatable independently of the endoscopic body portion of the instrument.

In another preferred embodiment, the surgical instrument includes an articulation member which can be pivoted about a pivot member positional transverse to the longitudianl axis of the body portion using an articulation link. An end effector preferably including a transducer is secured to the articulation member and pivotable with the articulation member in response to reciprocation of the articulation link to effect articulation of the end effector, i.e., vary the angle of the end effector in relation to the longitudinal axis of the instrument.

### BRIEF DESCRIPTION OF THE DRAWINGS

Various preferred embodiments of the presently disclosed ultrasonic surgical instrument are described herein with reference to the drawings, wherein:

FIG. 1 is a schematic representation of one embodiment of the presently disclosed ultrasonic surgical system including a surgical instrument for cutting, dissecting, ligating, coagulating and/or effecting hemostasis in tissue;

FIG. 1A is a side view of one preferred alternate embodiment of the ultrasonic member of the presently disclosed ultrasonic instrument;

FIG. 1B is a side view of another preferred alternate embodiment of the ultrasonic member of the presently disclosed ultrasonic instrument;

FIG. 1C is a side view of another preferred alternate embodiment of the ultrasonic member of the presentlu disclosed ultrasonic instrument;

FIG. 1D is a cross-sectional view taken along section lines X-X in FIG. 1C;

FIG. 1E is a cross-sectional view of an alternate embodiment of the ultrasonic member shown in FIG. 1D as would be seen along section line X-X of FIG. 1C;

FIG. 1F is a cross-sectional view of another alternate embodiment of the ultrasonic member shown in FIG. 1D as would be seen along section line X-X of FIG. 1C;

FIG. 1G is a cross-sectional view of yet another alternate embodiment of the ultrasonic member shown in FIG. 1D as would be seen along section line X-X of FIG. 1.

FIG. 1H is a top view of another alternate embodiment of the presently disclosed ultrasonic member;

FIG. 1I is a side perspective view of another embodiment of the presently disclosed ultrasonic member;

FIG. 1J is a side perspective view of another embodiment of the presently disclosed ultrasonic member;

FIG. 1K is a side view of another embodiment of the presently disclosed ultrasonic member;

FIG. 2 is a schematic top representation of one preferred embodiment of the ultrasonic member of the presently disclosed ultrasonic instrument;

FIG. 3 is a side view with portions broken away of the distal end of another preferred embodiment of the presently disclosed ultrasonic surgical instrument including an articulating end effector;

FIG. 4 is a top view with portions broken away of the distal end of the presently disclosed ultrasonic sugical instument shown in FIG. 3;

FIG. 4a is a top view with portions broken away of the distal end of the ultrasonic instrument shown in FIG. 4 in an articulated position;

FIG. 5 is a top view of a preferred embodiment of an ultrasonic member of the presently disclosed ultrasonic surgical instrument;

FIG. 6 is a side cross-sectional view with portions broken away of a proximal portion of another preferred embodiment of the presently disclosed ultrasonic instrument; and

FIG. 7 is a side cross-sectional view with portions broken away of the distal end of the ultrasonic instrument shown in FIG. 6.

### DETAILED DESCRIPTION OF PREFERRED EMBODIMENTS

Preferred embodiments of the presently disclosed ultrasonic surgical instrument will now be described in detail with reference to the drawings, in which like refence numerals designate identical or corresponding elements in each of the several view.

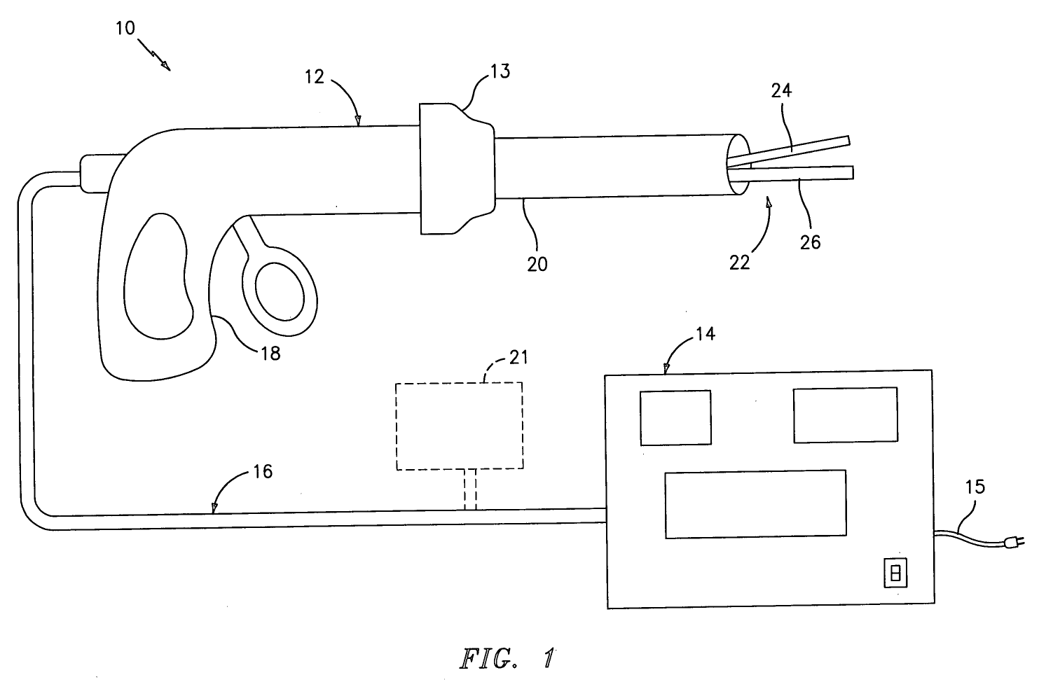

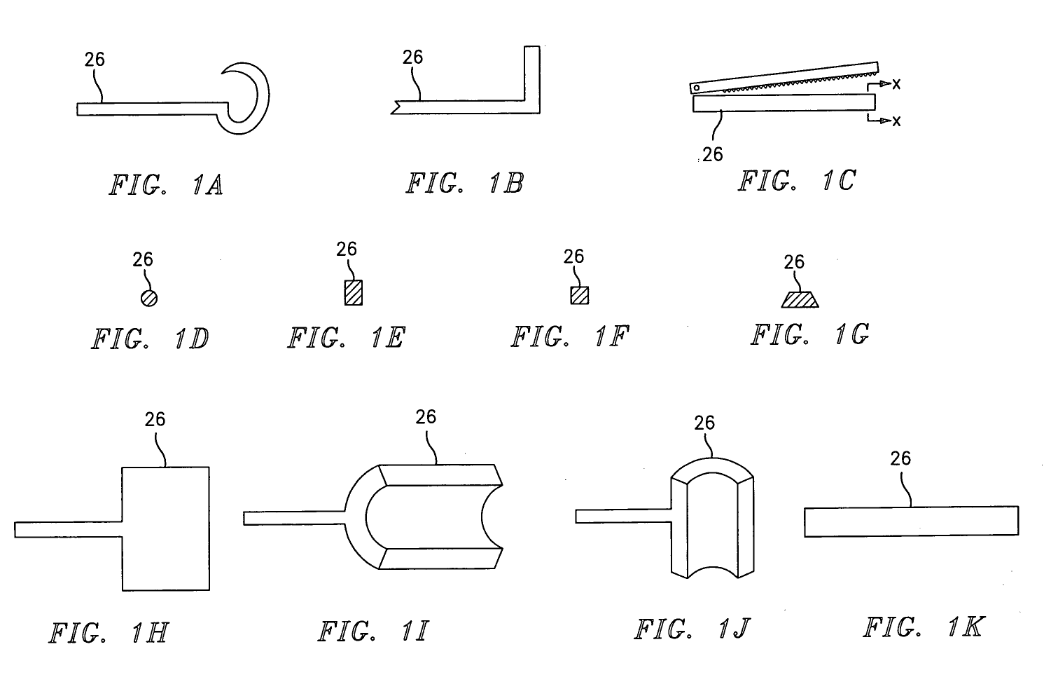

FIG. 1 illustrates a schematic view of an ultrasonic surgical system shown generally as 10. System 10 includes an ultrasonic instrument 12, a control module 14 and conductive cable 16 interconnecting instrument 12 to control module 14. Ultrasonic instrument 12 may be configured for open, endoscopic or laparoscopic sugical procedures and includes a handle assembly 18, an elongated body 20 and an end effector 22. Handle assembly 12 may have a pistol grip configuration, althrough other handle configurations are envisioned, e.g., in-line handle, pencil grips, standard scissor grips, new ergonomically designed grips, etc. Rotation knob 13 may be provided to facilitate rotation of elongated body 20 in known manner. End effector 22 includes a pivotable clamp member 24 and a linear ultrasonic member 26. Alternatively, the ultrasonic member of the end effectors may assume a variety of other configurations including, inter alia, J-hook (FIG. 1A), L-hook (FIG. 1B), shears (FIG. 1C) having a variety of different cross-sectional shapes (FIGS. 1D-1G), spatula (FIG. 1H), arcuate (FIGS. 1I AND 1J) and rectangular (FIG. 1K). The end effector may also be configured to have a curved blade such as the blade disclosed in U.S. Pat. No. 6,024,750, filed on Aug. 14, 1997 and/or an angled blade, such as disclosed in U.S. Pat. No. 6,036,667, filed on Oct. 4, 1996, both of which are incorporated herein in their entirety by reference.

The ultrasonic member may be formed using an etching process, e.g., isotropic etching, deep reactive ion etching, etc. Suitable etching processes are disclosed in U.S. Pat. No. 5,728,089 filed Oct. 31, 1994, which is also incorporated herein in its entirety by reference. Alternately, other known means may be used to form the ultrasonic member including a variety of different mechanical processes.

As illustrated, control module 14 may include a power cord 15 for engagement with an electrical outlet (not shown). Alternately, module 14 may be adapted to receive power from a battery pack or from an a/c generator. It is also envisioned that a generator or other power source may be incorporated into control module 14.

Module 14 includes electronic control circuitry to drive a transducer (not shown) positioned on instrument 12 at one or more ultrasonic frequencies. Protective circuitry is provided to prevent injury to a patient, a surgeon or system hardware. Module 14 also includes display circuitry and hardware to provide information to and accept information from a user. This information may be obtained from sensors (not shown) positioned on the instrument end effector. The sensors may be provided to monitor the temperature or, ultrasonic or electric impedence, of the tissue being operated on. Feedback circuitry may be provided to interact with any sensors provided to provide more effective ligation, cutting, dissection, coagulation, etc. For example, the feedback circuitry may terminate operation of the system if a sensor indicates that tissue temperature or ultrasonic or electrical impedence has exceeded a predetermined maximum. The ultrasonic impedence increases as tissue hardens due to rising temperatures. Similarly, electrical impedence is reduced when tissue water level is decreased due to overheating. The feedback circuitry may be selectively activated and deactivated and/or controlled or monitored by a surgeon to provide a surgeon more flexibility in operating the instrument. Further, control module 14 may include diagnostic circuitry to aid in testing and/or debugging instrument 12 or its hardware.

It is contemplated that operation of ultrasonic instrument 12 may be automatically controlled through the use of a computer. In one preferred alternative embodiment of the presently disclosed system, a computer 21 receives data from sensors positioned on the end effector of the ultrasonic instrument. As discussed above, sensors may be provided to monitor different characteristics of the tissue being operated upon including, inter alia, temperature and/or ultrasonic or electrical impedance. Computer 21 preferably includes circuitry to process an analogue signal received from the sensor(s) and to convert the analogue signal to a digital signal. This circuitry may include means to amplify and filter the analogue signal. Thereafter, the digital signal can be evaluated and operation of the ultrasonic instrument can be modified to achieve the desired effect in or on the tissue and prevent damage to surrounding tissue. Computer 21 may be incorporated into control module 14 or linked to control module 14 to effect the desired or appropriate modification of the operation of the instrument 12.

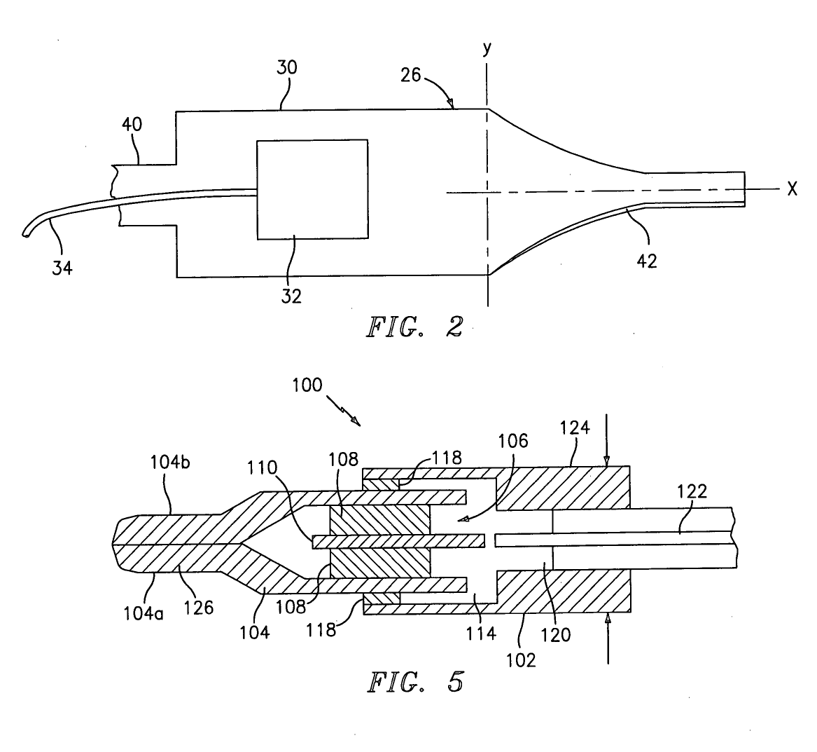

FIG. 2 illustrates a top or side schematic view of ultrasonic member 26 of an end effector 22. Ultrasonic member 26 includes a body portion 30 which is preferably formed of components made of silicon material. Alternately, materials such as titanium or other metals may be bonded or joined in some manner to the silicon to improve fracture resistance. It is envisioned that materials other than silicon which are suitable for ultrasonic use may be used to form ultrasonic member 26. A transducer 32, preferably a piezoelectric transducer, is supported on, or bonded to or within ultrasonic member 26. Piezoelectric transducer 32 is connected to the power source and control module 14 by an electrical connector, preferably a cable 34. Cable 34 may extend proximally from transducer 32 through body 20 of instrument 12 (FIG. 1) and exit instrument 12 through an opening (not shown) in the handle assembly 18 of the instrument.

As discussed above, ultrasonic member 26 may assume a variety of different configurations (FIGS. 1A-1K) and may be attached to a distal portion of instrument 12 in any known manner. For example, ultrasonic member 26 may be secured to a substrate or shaft or a mounting member (not shown) supported within a distal end of body 20 of instrument 12 such as by a snap-fit connection, a set screw or crimping or swaging. A threaded shank 40 or other attachment structure formed on or disposed on or in a proximal end of member 26 may be provided for attachment of ultrasonic member 26 to the distal end of instrument 12.

Transducer 32 can be positioned on or within or adjacent ultrasonic member 26 to effect vibration along any axis, e.g., the x-axis, the y-axis or any axis in between the x and y axis. Ultrasonic member 26 includes an operating surface generally designated 42 configured to effect dissection, cutting, coagulation, ligation and/or to effect hemostasis of tissue. Alternately, ultrasonic member 26 may include multiple operating surfaces to perform different tasks, e.g., cutting and coagulation. System 10, including instrument 12, can be used in a variety of surgical applications including general procedures, gynecologic, urologic, thoracic, cardiac and neurologic surgical procedures. Instrument 12 may be configured to perform both endoscopic and open surgical procedures and may be actuated via a finger switch or a foot pedal in a known manner. The actuation device may include wireless transmission circuitry to effect actuation of instrument 12.

By providing a transducer on, in or adjacent the distal tip of the instrument, the following benefits can be realize: a) the need for an elongated vibration coupler formed of titanium is obviated substantially reducing the cost of the instrument; b) the length of the body portion of the instrument can be changed, e.g., shortened or lengthened, with virtually no consequential change in instrument performance, e.g., since the instrument vibration coupler has been replaced by an electrical conductor, the instrument need not be retuned, at considerable expense, after changes in body length; c) ultrasonic energy can be transferred to a patient more efficiently, thus lowering energy power requirements; d) the portion of the instrument that is disposable can be easily varied and may comprise only the instrument tip with a limited reuse handle, the entire instrument or any degree of disposability therebetween; e) because the handle assembly does not support the transducer, the handle assembly can be more ergonomically configured; and f) the use of a small transducer on, in or adjacent the distal end of the instrument in place of a large transducer on the proximal end of the instrument substantially reduces the weight of the instrument and makes it easy to manage especially during delicate surgical procedures.

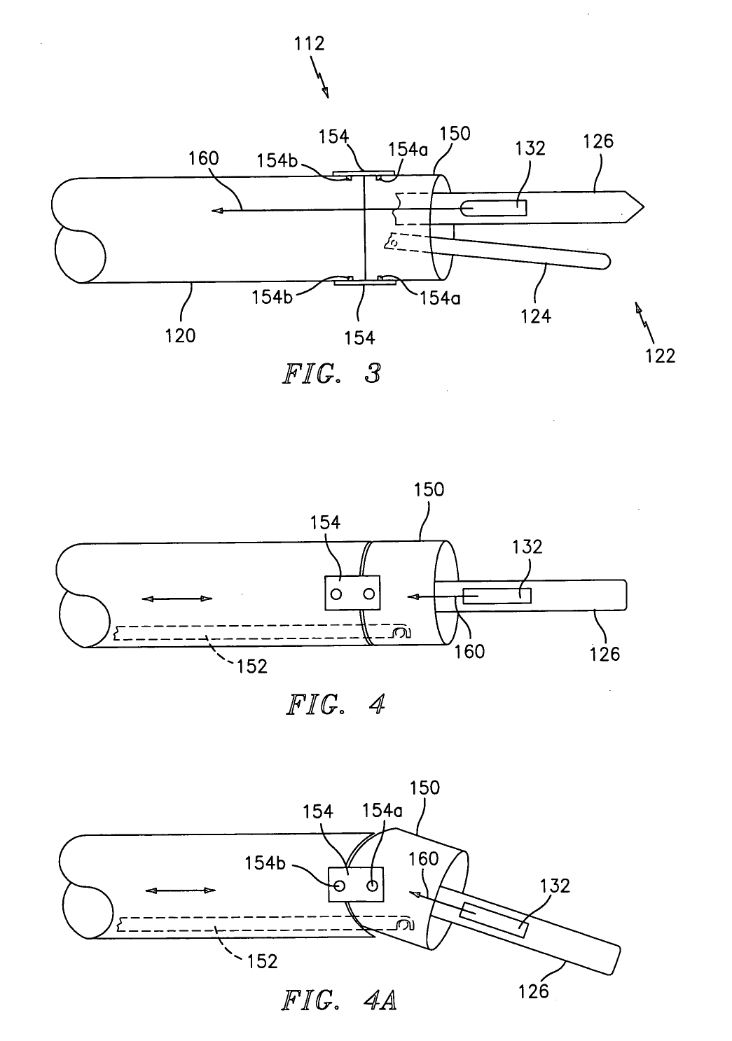

FIGS. 3 and 4 illustrate the distal end of another preferred embodiment of the presently disclosed ultrasonic surgical instrument shown generally as 112. Instrument 112 includes an end effector 122 having an ultrasonic member 126 and a clamping jaw 124, a body portion 120 defining a hollow throughbore, an articulation member 150 and an articulation link 152 (FIG. 4). Ultrasonic member 126 includes a transducer 132. Preferably, the transducer is located as close to the distal end of ultrasonic member 112 as possible. A wire 160 interconnects transducer 132 to a power source (not shown). End effector 122 is supported within articulation member 150 and articulation member 150 is pivotably supported by members 154 about projections 154a to body portion 120. Articulation link 152 has a distal end which is pivotably connected to articulation member 150 at a location offset from pivot members 154. Articulation link 152 is linearly movable within body 120 to pivot member 150 about projections 154 to effect articulation of end effector 122. Articulation member 150 may be configured to effect articulation over an angle of between 5° and 175° and preferably between 30° and 120°. Because transducer 132 is supported on ultrasonic member 126 of end effector 122, end effector 122 of ultrasonic instrument 112 can be articulated without interfering with the vibratory operation of the ultrasonic member (See FIG. 4A.)

FIG. 5 illustrates one preferred embodiment of an ultrasonic member, shown generally as 100, suitable for use in the presently disclosed ultrasonic surgical instrument of ultrasonic surgical system 10. Ultrasonic member 100 is preferably a piezoelectric laminate structure which includes a frame 102, a resonant structure 104, and a transducer 106. Alternately, other transduction mechanisms, other than piezoelectric may be used including thermal stress, electrostriction, magnetostriction or optical drive mechanisms. Transducer 106 preferably includes a pair of PZT crystals 108 separated by silicon plate 110. Alternately, it is envisioned that crystals other than PZT crystals may be used to convert electrical power to effect mechanical vibration. An appropriate bonding agent or process, e.g., solder bonding, diffusion bonding, adhesives, etc., is used to fasten crystals 108 to plate 110. Resonant structure 104 is preferably formed from a silicon or metal resonant structure or a silicon/metal composite. Structure 104 preferably includes first and second resonant members 104a and 104b. The proximal end of members 104a and 104b together define a cavity for receiving transducer 106. Alternately, resonant structure 104 may be monolithically formed from a single piece of material. The mating surfaces of PZT crystals 108 and resonant members 104a and 104b are fastened together using an appropriate bonding agent or bonding process, e.g., glass binding, adhesives, etc. Frame 102 includes a body 112 which is preferably formed from a rigid material including metals, ceramics, etc. and includes a cavity 114 dimensioned and configured to receive the resonant structure 104 and transducer 106 assembly. A bonding layer or layers 118, preferably formed of a conductive material, is positioned between the proximal portion of resonant members 104a and 104b and frame 102 to bond transducer 106 which is movable to frame 102 which is stationary. The proximal end of frame 102 includes a throughbore 120 which is dimensioned to permit passage of an electrical conductor 122, e.g., a wire or coaxial cable, to provide power to transducer 106. The electrical conductor is preferably a high-voltage high-frequency Teflon insulator cable, although the use of other conductors is envisioned. The distal end of conductor 122 is connected to plate 110 by a flexible conductive wire 124 which does not restrict relative movement between frame 102 and transducer 106.

As discussed above, the shape of resonant structure 104 may be different than that shown in FIG. 5. More specifically, distal operating surface 126 resonant sturcture 104 may assume any of the configurations shown in FIGS. 1A-1K or any other configuration not shown herein which may be advantageous for performing a particular surgical procedure. Moreover, a clamp may be provided to facilitate gripping of tissue.

Ultrasonic member 100 can be actuated in both high and low frequency ranges. In the low frequency range, approximately 20-100 KHz, the instrument will cause cavitation in tissue to effect cutting of the tissue. In the high frequency range, greater than 1 MHz, the instrument may be used for heating and coagulation of tissue. The high and low frequency actuation may occur sinultaneously by an electronic power amplifier, capable of generating both frequencies. Providing multiple frequencies may provide improved cutting in tissue with reduced thermal spread and improved coagulation and hemostasis.

As discussed above, power losses and inefficiencies are substantially reduced as compared to conventional ultrasonic instruments by placing the ultrasonic energy generating PZT element adjacent, on or within the ultrasonic member of the end effector. Whereas conventional instruments may require 40-50 watts of electrical power to effect cutting of tissue, it is envisioned that the presently disclosed ultrasonic instrument will require only 20-30 watts of electrical energy to effect the cutting of tissue. Moreover, it is envisioned that the presently disclosed laminated structure of ultrasonic member 100 is operable at higher frequencies than conventional instruments. Because it is believed the use of higher frequencies may speed the rate of coagulation at a given power setting, the power requirements may be further reduced by operation of the instrument at higher frequencies.

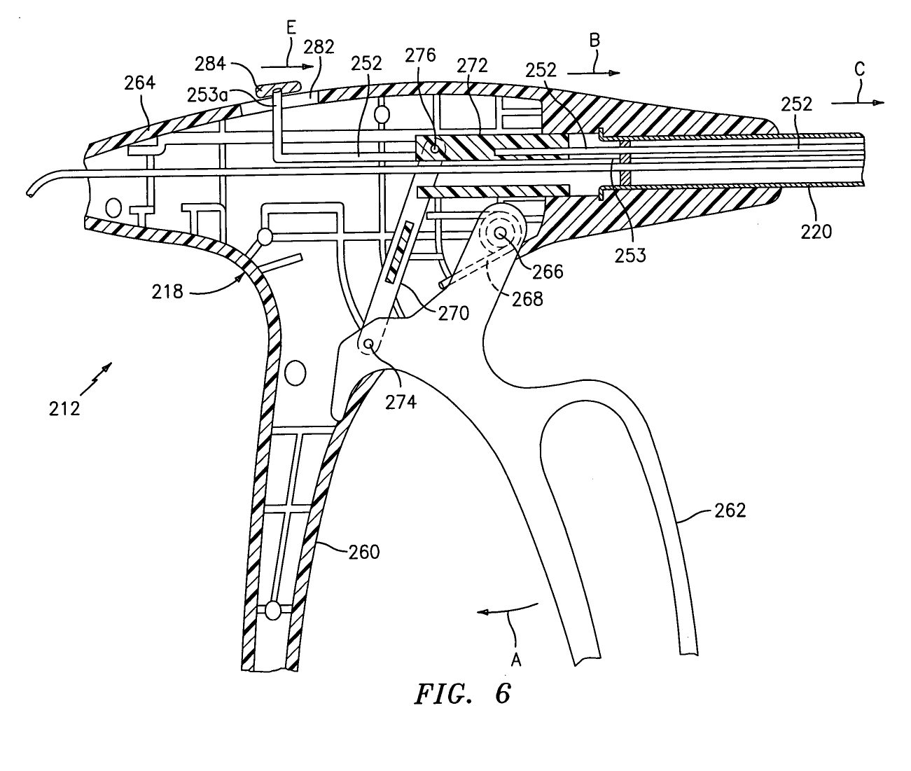

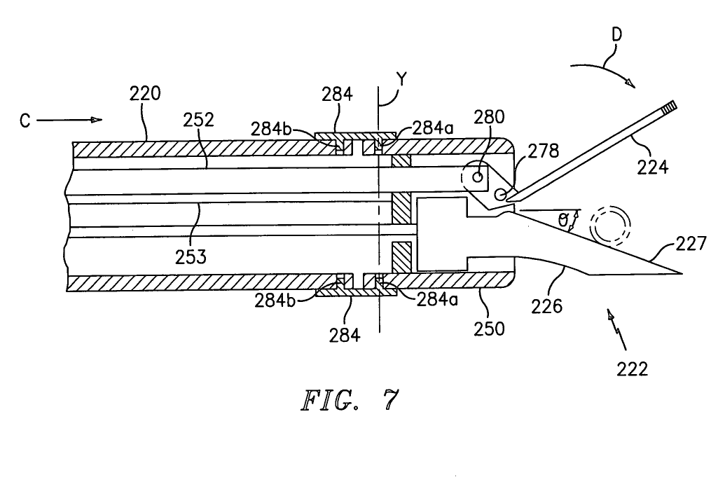

FIGS. 6 and 7 illustrate another preferred embodiment of the presently disclosed ultrasonic instrument shown generally 212. Ultrasonic instrument 212 includes a handle assembly 218 (FIG. 6), an elongated body 220 and an end effector 222 (FIG. 7). Handle assembly 218 includes a stationary handle portion 260 and a pivotable handle portion 262. Pivotable handle 262 is pivotably mounted to body portion 264 of handle assembly 218 about a pivot member 266 and is movable from a non-actuated position (FIG. 6) to an actuated position by moving handle 262 towards handle 260 against the bias of biasing member 268 in the direction indicated by arrow “A” in FIG. 6. A link 270 translates the pivotable movement of handle 262 to a linear drive member 272. Link 270 has a first pivotably secured to pivotable handle 262 by a pin 274 and a second end pivotably secured to drive member 272 by a pin 276. Upon movement of pivotable handle 262 to the actuated position, linear drive member 272 moves in the direction indicated by arrow “B” in FIG. 6.

A flexible clamping rod or link 252 has a proximal end secured to drive member 272. Clamping link 252 is preferably formed of a shape memory or resilient material and has a distal end connected to a pivotable clamp member 224 (FIG. 7). Clamp member 224 is pivotably secured within a mounting member 250 by a pivot member 278. The distal end of clamping link 252 is pivotably connected to pivotable clamp member 224 by a pin 280 at a location offset from pivot member 278. In use, when handle 262 is moved in the direction indicated by arrow “A” (FIG. 6) to move drive member 272 in the direction indicated by arrow “B”, clamp link 270 is advanced distally in a direction indicated by arrow “C” in FIGS. 6 and 7. Distal movement of clamp link 270 pivots clamp member 224 about pivot member 278 in the direction indicated by arrow “D” in FIG. 7 to a clamped position in juxtaposed alignment with ultrasonic member 226.

As illustrated in FIG. 6, an articulation link 253 is slidably positioned within body portion 264 of handle assembly 218. Link 253 includes a proximal end 253a which extends through a slot 282 formed in body portion 264. A slide member 284 is secured to proximal end 253a of link 253 and is movable along the outer surface of body portion 264 in the direction indicated by arrow “E” to effect distal movement of articulation link 253.

Referring to FIG. 7, a mounting member 250 is pivotably secured to the distal end of elongated body 220 by pivot members 284. Pivot members 284 each include first and second projections 284a and 284b, respectively. Projections 284a are pivotably secured to elongated body 220 and projections 284b are pivotably secured to mounting member 250 such that mounting member 250 is pivotable with respect to elongated body 220 about a transverse axis Y. The distal end of articulation link 253 is engaged with a projection (not shown) extending outwardly from an inner surface of mounting member 250. The projection is laterally offset from pivot axis Y. When link 253 is moved distally or proximally, mounting member 250 is pivoted about pivot axis Y to an articulated position. See FIG. 4A. In a preferred embodiment, mounting member 250, and thus end effector 222, can be articulated over an arc of about 150°.

End effector 222 includes clamp member 224 and ultrasonic member 226. Ultrasonic member 224 is secured within mounting member 250 using any known fastening technique including crimping, swaging, screws, etc. Ultrasonic member 224, although shown schematically, is substantially the same as ultrasonic member 100, except operating surface 126 includes a blade configuration. As discussed above, when mounting member 250 is pivoted about axis Y by articulation link 253, end effector 222 including ultrasonic member 224 are also pivoted, i.e., articulated, about transverse axis Y.

It will be understood that various modifications may be made to the embodiments disclosed herein. For example, the configuration of the ultrasonic member of the end effector need not be as shown herein but rather may be modified to suit a particular surgical application. Further, the transducer may be mounted proximally of the ultrasonic member of the end effector in the distal end of the instrument and need not be mounted directly to the ultrasonic member. Therefore, the above description should not be construed as limiting, but merely as exemplifications of preferred embodiments Those skilled in the art will envision other modifications within the scope and spirit of the claims appended hereto.

What is claimed:

1. A surgical instrument comprising:
a handle;
an elongated body portion extending distally from the handle, the elongated body being dimensioned and configured to pass through a cannula or body orifice; and
an end effector supported on the distal end of the elongated body portion, the end effector including an ultrasonic member having a transducer and a resonant member, the resonant member being operatively connected to the transducer and including an operating surface configured to effect tissue dissection, cutting, coagulation, ligation and/or hemostasis.

2. A surgical instrument according to claim 1, wherein the end effector includes a pivotable clamp, the pivotable clamp being movable in relation to resonant member of the ultrasonic member between an open position spaced from the operating surface of the resonant member and a closed position in juxtaposed alignment with the operating surface of the resonant member.

3. A surgical instrument according to claim 1, wherein the ultrasonic member has a J-hook configuration.

4. A surgical instrument according to claim 1, wherein the ultrasonic member has an L-hook configuration.

5. A surgical instrument according to claim 1, wherein the ultrasonic member has a rectangular configuration.

6. A surgical instrument according to claim 1, wherein the ultrasonic member has a spatula configuration.

7. A surgical instrument according to claim 1, wherein the transducer includes a plurality of PZT crystals positioned about a silicon plate.

8. A surgical instrument according to claim 7, wherein the resonant member is formed from a silicon/metal composite.

9. A surgical instrument according to claim 1, further including an articulation member pivotably attached to the distal end of the elongated body portion, the end effector being supported by the articulation member, wherein the articulation member is pivotable to effect articulation of the end effector.

10. A surgical instrument according to claim 1, further including a sensor positioned on the ultrasonic member for monitoring a condition of tissue to be operated upon.

11. A surgical instrument according to claim 10, wherein the condition is temperature.

12. A surgical instrument according to claim 10, wherein the condition is ultrasonic or electrical impedence.

13. An ultrasonic surgical system comprising:
an endoscopic ultrasonic instrument including a handle, and elongated body portion and an end effector, the end effector including an ultrasonic member having a transducer and the elongated body portion being dimensioned and configured to be insertable through a cannula or body orifice;
a control module adapted a engage a power source, the control module including electronic control circuitry to drive the transducer at one or more ultrasonic frequencies; and
an electrical cable interconnecting the control module and the ultrasonic instrument.

14. An ultrasonic surgical system according to claim 13, further including an electrical conductor positioned within the ultrasonic instrument having a distal end communicating with the transducer and a proximal end adapted to engage the electrical cable.

15. An ultrasonic surgical system according to claim 14, wherein the electrical conductor includes coaxial cable.

16. An ultrasonic sugical system according to claim 15, further including a sensor positioned on the ultrasonic member to sense characteristics of tissue being operated upon, and wherein the control module includes feedback circuitry which interacts with the sensor to control operation of the ultrasonic instrument.

17. An ultrasonic surgical system according to claim 13, wherein the end effector is supported on the surgical instrument for articulation.

18. An ultrasonic surgical system according to claim 13, wherein the ultrasonic member includes a frame, resonant structure and the transducer, wherein the transducer is positioned in contact with the resonant structure and the resonant structure includes an operating surface for effecting dissection, cutting, ligation and/or coagulation of tissue.
